(19) United States
(12) Patent Application Publication
(10) Pub. No.: US 2024/0058815 A1
(43) Pub. Date: Feb. 22, 2024

(54) DEVICE FOR THE DETECTION OF AT LEAST ONE ANALYTE IN A SAMPLE

(71) Applicant: digid GmbH, Mainz (DE)

(72) Inventors: Nils KÖNNE, Mainz (DE); Constantin VON GERSDORFF, Mainz (DE); Konstantin KLOPPSTECH, Mainz (DE)

(21) Appl. No.: 18/236,655

(22) Filed: Aug. 22, 2023

(30) Foreign Application Priority Data
Aug. 22, 2022 (DE) 102022121185.1

(51) Int. Cl. B01L 3/00

(52) U.S. Cl. CPC B01L 3/502715; B01L 2300/0663; B01L 2400/0406; B01L 2300/123; B01L 2300/023; A61B 10/0045

(57) ABSTRACT

A device is provided for the detection of an analyte in a sample. The device includes at least one capillary, at least one measurement chamber and at least one cantilever pair. The capillary is configured to take a sample and to feed it to the measurement chamber that collects the sample and provides it to the cantilever pair. The cantilever pair includes a reference cantilever and a test cantilever. The test cantilever selectively uptakes the analyte from the sample and the reference cantilever selectively non-uptakes the analyte from the sample. The respective uptakes cause a relative deformation and/or a relative change in the surface tension of the test cantilever relative to the reference cantilever. The relative deformation and/or the relative change in surface tension of the test cantilever and the reference cantilever results in detection of the analyte.

### CROSS REFERENCE TO RELATED APPLICATIONS

This application claims priority to German Patent Application No. 10 2022 121 185.1, filed Aug. 22, 2023, the entire contents of which are hereby incorporated by reference in their entirety.

### TECHNICAL FIELD

The present invention relates to a device for detection of at least one analyte in a sample.

### BACKGROUND

The utilization of spring elements or cantilevers for detection of analytes in samples is known. In this case, the interaction of cantilevers with a sample liquid and the binding of the analyte in the sample to a coating of the cantilever is exploited in order to cause deformation or a change in surface tension of the cantilever. The deformation or change in surface tension can then be used to infer, via a strain gauge, the deformation of the cantilever by the analyte and hence indirectly the occurrence of the analyte.

The deformation of cantilevers of different surface tensions is described, for example, in Rasmussen, P. A., Hansen, O., & Boisen, A. (2005) “Cantilever surface stress sensors with single-crystalline silicon piezoresistors” Applied Physics Letters, 86(20), 203502. https://doi.org/10.1063/1.1900299.

However, the detection of an analyte in a sample may be found to be difficult because of large mechanical disruptive influences by the sample liquid and owing to other chemical substances in the sample liquid, such that it is necessary to extract the components from the actual measurement signal that are attributable solely to the interaction of the analyte with the cantilever.

Moreover, if the analyte is to be detected regularly in a body fluid, for example for permanent examination of a patient on for monitoring of their body values, a device is required that can firstly supply new sample liquid in order to enable time resolution of the body values, and secondly can be secured to the patient without an operation if at all possible.

EP 297 226 4 and U.S. Pat. No. 2,021,321 942 disclose wearable plasters comprising microneedles that penetrate through the skin and where various analytes from the interstitial liquid can be determined by electrochemical measurements.

EP 185 080 3 and WO 111 433 24 disclose implantable sensors that detect the deformation of functionalized cantilevers on interaction with an analyte.

### SUMMARY

In view of the foregoing, it is an object of the present invention to provide an improved device for detection of an analyte in a sample.

The object is achieved by a device for the detection of an analyte in a sample having the features of claim 1. Advantageous embodiments will become apparent from the dependent claims, the description and the figures.

Accordingly, a device for the detection of at least one analyte in a sample is proposed, comprising at least one capillary, at least one measurement chamber and at least one cantilever pair, where the capillary is configured to take a sample and to feed it to the measurement chamber, where the measurement chamber is configured to collect the sample and provide it to the cantilever pair, and where the cantilever pair comprises a reference cantilever and a test cantilever, where the test cantilever is configured for selective uptake of the analyte from the sample and where the reference cantilever is configured for selective non-uptake of the analyte from the sample, where the selective uptake of the analyte by the test cantilever and the selective non-uptake by the reference cantilever causes a relative deformation of the test cantilever relative to the reference cantilever, and where the relative deformation of the test cantilever and the reference cantilever results in detection of the analyte.

A sample here refers to a limited amount of a substance that has been taken from a greater amount of the substance, for instance from a reservoir, where the composition of the sample is representative of the composition of the substance in the reservoir and the corresponding occurrence in the reservoir can accordingly be inferred from the occurrence of the substance in and the substance compositions of the sample.

For example, a sample may also be a body fluid, especially a cellular fluid or an interstitial fluid or a blood sample or perspiration. The sample may also be or comprise a lymph fluid or lymph. A sample may alternatively be a non-biological sample, for example a sample of a chemical substance. The sample contains the chemical information and/or the biochemical information about the analyte.

An analyte here is the substance the occurrence of which in the sample is to be detected qualitatively and/or quantitatively or to be detected with the cantilever pair. The analyte may in particular be present directly in the sample, or be dissolved in the sample, or adhere to the sample or to part of the sample, in particular to a sample particle. The analyte may also enter into a chemical, biological and/or physical interaction with the sample, such that the analyte is detectable only indirectly via a corresponding interaction with the cantilever pair, especially with the test cantilever. For example, a virus can be digested chemically and/or biologically and/or physically such that interaction of a resulting analyte with the cantilever pair, especially the test cantilever, is possible.

The chemical information may include, for example, the type of the analyte, the concentration of the analyte, the occurrence of the analyte, the weight of the analyte, the reactivity of the analyte, the density of the analyte, etc. The biochemical information comprises the same properties as the chemical information, except that these substances may arise, for example, from biological processes. In particular, reference is made to biochemical information when the analyte has a particular effect on the biological cycle, for example metabolism or the immune system.

In other words, the analyte may be a biomarker and the sample liquid may be a body fluid.

The at least one capillary may be configured to take the sample from the sample body.

For example, the capillary may be cylindrical or run to a point and be hollow in shape or take the form of a straw. In particular, the capillary may take the form of a needle or microneedle. The capillary may preferably be of a stable form in order to prevent breakage in or on the sample body.

For example, the capillary can take the sample liquid from the sample body at a constant flow rate. The flow rate can be adjusted in a particularly simple manner, for example, via the diameter of the capillary.

In addition, the capillary may be coated in order to reduce adhesion of the sample liquid or of chemical species therein or in order to reduce friction with the sample body. The coating can additionally adjust the surface energy of the capillary in order, for example, to create a hydrophobic or hydrophilic coating, or a mixture thereof. It is preferably thus also possible to avoid or slow protein deposition in or on the capillary.

The capillary can penetrate by its open end into a sample body or sample reservoir and from there transport the sample liquid to its second end. The sample liquid can then be collected in the measurement chamber.

For this purpose, the capillary may have a diameter of less than 200 μm, preferably of less than 100 μm, and have a length between 500 μm and 5000 μm.

When the device is used for the first time, the measurement chamber has to be filled with sample liquid. One way of doing this is to fill the measurement chamber by (repeatedly) pressing the device onto the sample body. For this purpose, the device is preferably correspondingly formed mechanically such that uniform pressing onto the sample body can be achieved.

By virtue of the small diameter of the capillary, it is possible for the capillary to enter the sample body in a particularly simple manner. The length of the capillary simultaneously enables entry of the capillary into the sample body with sufficient depth that the sample liquid can be transported away in a sufficient amount. In particular, depending on the sample body, it is possible via the length of the capillary to control what sample liquid should be taken. For example, shorter capillaries can take an interstitial fluid from a human sample body, whereas longer capillaries can take blood from the sample body.

The capillary can feed the sample to the measurement chamber especially via diffusion forces and/or osmotic forces and/or capillary forces.

The capillary force is based here on the surface tension between sample liquid and capillary material. This makes it possible for the sample liquid to be taken through the capillary counter to gravity without expenditure of an additional external force.

The capillary force may be adjusted via a specific coating of the capillary, and in this way the flow through the capillary can be adjusted. The diffusion force here generates a directed flow of particles or liquid, which attempts to compensate for the concentration gradient of a substance between two connected reservoirs.

For example, the diffusion force can generate a constant stream of particles when the concentration of the analyte on one side of the capillary, for example in a sample depot, on account of its volume (see below), is always kept low or at zero. As a result, the diffusion gradient always attempts to balance out the concentration gradient of the analyte, in that the concentration in the measurement chamber always corresponds to the concentration in the sample body.

Because the capillary connects the sample body to the measurement chamber, the concentration gradient of the analyte builds up a diffusion potential that results in a flow of particles. In addition, the sample liquid is guided through the capillary by the diffusion force, which can be assisted, for example, by a nanoporous sample depot (see below) as capillary pump. The diffusion force is thus suitable for guiding small amounts of sample liquid into the measurement chamber without actively conducting a pumping operation. The capillary may have a constant internal diameter or have a conical internal profile and may especially narrow in the direction of the measurement chamber volume. The capillary may preferably prevent reflux of the liquid from the measurement chamber volume into the sample via specific adjustment of the diameter.

Additionally or alternatively to the above mentioned mechanisms, the sample liquid can also be moved through the capillary by convection, drift or an externally introduced force, for example an electrical potential or a magnetic field.

The sample liquid is transported through the capillary to the measurement chamber. The measurement chamber here may enclose a measurement volume, where the measurement volume is limited by a sample inlet area having at least one capillary, at least one measurement chamber wall adjoined thereto, and a measurement chamber lid.

The measurement chamber wall can form an angle here with the sample inlet area, such that the measurement chamber has a certain volume. The measurement chamber layered can bound said measurement chamber volume and keep the sample liquid within the measurement chamber volume. It is also possible for the measurement chamber to be semipermeable, such that the sample liquid can leave the measurement volume again after a certain time. The measurement chamber wall may preferably also be nanoporous and guide the sample liquid away in the outward direction.

It is also possible to provide more than one measurement chamber, in which case at least one cantilever pair or individual cantilevers are arranged in each measurement chamber. The providing of more than one measurement chamber allows the sample liquid conveyed through the capillary into the measurement chamber to be treated differently, for example treated chemically and/or biologically and/or physically, in order then to digest the corresponding analyte(s) which then interact(s) with the cantilever pair, especially the test cantilever. The providing of more than one measurement chamber can accordingly achieve the effect that different treatments of the sample liquid can be conducted, and in this way a multitude of analytes can be characterized.

For conversion of the chemical and/or biochemical information from the analyte to an electrical signal, the device comprises a reference cantilever and a test cantilever that form a cantilever pair. A cantilever here is a spring element that has a base and a deformable part.

The base here is an immobile part of the cantilever which is especially fixedly bonded to the substrate and/or is supported by and/or has been elaborated from the substrate. The base of the cantilever takes the form of a rigid base, such that only the deformable part of the cantilever is deformable.

The deformable part of the cantilever extends in longitudinal direction beyond the substrate on which the base is arranged. In other words, the deformable part of the cantilever is suspended on the base at one end and is not supported by the substrate. Because the deformable part projects beyond the substrate, the deformable part of the cantilever can be bent, deflected and extended. The spatial limit from which the cantilever is bendable or the cantilever transitions from the base to the deformable part is called the bending edge. The bending edge is usually an edge of the substrate when the cantilever protrudes beyond the substrate.

If the cantilever is deformed, this results in material stresses and forces in or on the cantilever material that can be measured. If such a material stress and/or force can be measured, it is possible to infer a deformation of the cantilever therefrom. A deformation may be a lifting or lowering deformation. The cantilever may alternatively itself deform, for example bend, corrugate or distort.

The deformation of the cantilevers can be brought about in a substance-specific manner by means of a suitable coating. For that reason, the reference cantilever has a reference layer for selective non-uptake of the analyte, whereas the test cantilever has a receptor layer for uptake of the analyte.

A receptor layer here is a substance that can interact with the analyte. This in turn means that the receptor layer is chosen specifically for each analyte. Analogously, a reference layer is a substance that cannot interact with the analyte. The reference layer too is therefore chosen specifically for the analyte.

Interaction means that the analyte is in chemical and/or biochemical and/or physical interaction with the receptor layer. In particular, the interaction may consist in binding of the analyte to the receptor layer. An interaction may also consist in the absorption or adsorption of the analyte to the receptor layer.

The receptor and reference layers are preferably chemically identical with regard to possible disruptive influences, and preferably differ solely by the interaction with the analyte. A substance that is not the analyte accordingly interacts just as strongly or just as weakly with the receptor layer as with the reference layer.

The effect of the selective uptake of the analyte on the test cantilever is that a force acts on the test cantilever as a result of the analyte, such that the test cantilever is sensitive to the analyte. Accordingly, the other substances in the sample that are not the analyte contribute merely to background noise in the form of background deflection of the test cantilever. A maximum force possible for the respective form is achieved when the cantilever is fully covered.

By contrast, the effect of the selective non-uptake of the analyte on the reference cantilever is that no force acts on the reference cantilever as a result of the analyte, such that only the substances that are not the analyte contribute to background noise in the form of background deflection of the reference cantilever.

The force acting on the test cantilever via the adsorption of the analyte can bring about deformation in the deformable part of the test cantilever, while the deformable part of the reference cantilever is not deflected. The reason for the deflection of the cantilever is the change in surface tension caused by the interaction with the analyte. The change in surface tension leads to extension or contraction of the upper (or lower) surface of the cantilever. The different extension or contraction on the upper and lower sides results in an internal force or material stress in the material that leads to deformation.

Reference cantilevers according to the prior art merely have no receptor layer which is sensitive to the analyte. It is possible in this way to determine effects such as turbulence in the sample and thermal drift, and also pH and variations in concentration of the ion concentration in the sample liquid, of the sensor system. However, in such a reference cantilever, the analyte can, for example, bind to the reference layer of the reference cantilever through non-specific binding. However, the analyte itself thereby contributes to the background noise. Therefore, in the case of a sensor according to the prior art, there is a need for reference measurements in a reference sample, i.e. a sample without analytes. Only in this way can the effect of the non-specific binding of the substances that are not the analyte be established.

In the device according to the invention, the selective non-uptake of the analyte by the reference cantilever significantly simplifies the test method, since the reference cantilever is not sensitive to the analyte, and therefore the analyte does not contribute to the background noise either. Only the substances that are not the analyte contribute here to the background noise of the reference cantilever. The selective non-uptake of the analyte on the reference cantilever may be brought about to some degree in that the reference cantilever is exposed to the same turbulences, the same thermal drift and the same influence of all substances that are not the analyte as in a reference liquid. The difference is, however, that the reference signal is determined directly in the sample liquid.

In particular, a reference cantilever with reference layer and a test cantilever with receptor layer results in distinctly more specific analysis of the analyte than just a reference cantilever without receptor layer, since both the reference layer and the receptor layer have a specific interaction or non-interaction with the analyte.

The structure of the sensor with reference cantilever and test cantilever has the advantage that two measurements can be undertaken simultaneously in the sample, and the measurement by the reference cantilever can calibrate the measurement by the test cantilever. This makes it possible to reduce environmental influences, for instance chemical, thermal, mechanical, electrical and fluidic disruptive influences, on the respective measurement, such that occurrence of the analyte can be inferred from the comparison of the measurement on the test cantilever and on the reference cantilever.

These forces or material stresses, for example extensions or contractions, that act on the cantilever can ultimately be detected by transducers, and stresses of different magnitude are detected by the transducer through extensions or contractions of different magnitude.

The transducers accordingly have the purpose of determining or of measuring the deformation of the cantilevers.

The transducers are preferably arranged on the deformable parts and/or the bases of the cantilevers. For example, a deformation of the cantilever may have the effect that the resistance of a transducer rises or falls, whereas, by contrast, no deformation of the cantilever causes no change in the resistance of the transducer either. This can be achieved, for example, via a configuration of the transducers in accordance with the principle of a strain gauge, as a result of which a deformation of the respective cantilever is manifested in a change in length of the transducer strain gauge applied thereto, and hence it is possible to detect a deformation of the cantilever directly by a change in the resistance of the strain gauge.

The chemical and/or biochemical information from the analyte is thus detectable via a deformation of the cantilever, subsequent registration via a transducer, and finally via a change in an electrical property of the transducer.

Because the first and second test transducers, for example, are arranged on the deformable parts of the test cantilever and the first and second reference transducers are arranged on the deformable part of the reference cantilever, it is possible to find a measure of extension of the test transducer that corresponds to the strength of the interaction of the analyte with the deformable part of the test cantilever.

Because the first test transducers, for example, are arranged on the deformable parts of the test cantilever, second reference transducers are arranged on the base on the test cantilever, first reference transducers are arranged on the deformable part of the reference cantilever and second reference transducers are arranged on the base of the reference cantilever, it is possible to find a measure of extension of the first test transducer that corresponds to the strength of the interaction of the analyte with the deformable part of the test cantilever.

For example, the first reference transducer of the reference cantilever, influenced by the environmental conditions and interacting with the sample, can cause a first electrical reference state of the first reference transducer, while the interaction of the test cantilever with the environmental conditions of the sample causes a first electrical test state of the first test transducer.

For example, the reference cantilever may be bent by a first amount as a result of the influence of the ambient conditions, such that the deflection causes a first reference state in the first reference transducer and causes a second electrical reference state in the second reference transducer, while the test cantilever is bent by a second amount as a result of the influence of the ambient conditions and is bent by a third amount as a result of the additional interaction with the analyte in the sample, which causes a first electrical test state in the first test transducer and a second electrical test state in the second test transducer.

Comparison of the electrical states of the first and second transducers gives a measure of the deformation of the cantilevers. At the same time, comparison of the respective first transducers and/or of the respective second transducers gives a measure of the difference in deformation of the cantilevers. This makes it possible to infer a specific influence of an analyte on the test cantilever, preferably to quantify the influence and hence to infer a concentration-dependent influence.

The detection of the analyte in the sample can be conducted continuously. In other words, there is not just a single detection of the analyte; instead, the detection is conducted over a period of time. The concentration is preferably conducted in a time-resolved manner, such that changes in the presence and/or concentration of the analyte can be detected over the period of time.

This is in particular the case when sample liquid is added to the measurement volume and the analyte has only a limited binding time at the test cantilever, which can be adjusted via the choice of test coating. After the binding time, the analyte becomes detached from the test coating, such that the corresponding binding site can be taken by the analyte of the subsequent sample liquid.

It is also possible that the test coating has a capacity of binding sites that are occupied ever further by the analyte of the incoming sample with time. Accordingly, it would be possible for the cantilever to bend ever further until all the binding sites are bound. It is then possible to infer the analyte from the change in bending with time or the instantaneous bending.

This has the advantage that the analyte, or the occurrence thereof, can be analysed in a time-dependent manner. This is of interest, for example, when particular vital values in the sample body are to be examined, for example lactate or glucose.

For example, it is possible to analyse the analyte by a continuous measurement without consuming it chemically, especially converting it chemically.

The cantilever size and hence the possible area of interaction with the analyte can be adjusted to the flow rate through capillary. For example, it is possible to reduce the effect of small fluctuations in flow rate on the measurement.

The cantilever pair may be arranged on or in the measurement chamber wall.

Arranging the cantilever pair on the measurement chamber wall can achieve the effect that the cantilevers and the measurement chamber are separated from one another in terms of construction, such that the two components can be manufactured independently. In particular, this can achieve the effect that the cantilevers are spaced apart with respect to the sample inlet area of the measurement chamber, such that the inflow of the sample liquid to the cantilever pair has only a minor flow-related effect on the detection of the analyte, if any. The distance corresponds here roughly to the height of the measurement chamber wall.

If the cantilever pair is arranged in the measurement chamber wall, the device can be made particularly compact.

The measurement chamber may have a sample depot as or on the measurement chamber wall, especially a nanoporous reservoir configured to take up a sample.

The uptake of the sample can ensure, for example, that the sample liquid is stored. As a result, in particular, the sample liquid can leave the measurement chamber volume accessible to the cantilevers after a certain time, such that the measurement chamber volume can be filled with a new sample liquid. The sample depot can preferably function as a capillary pump, and hence minimize the concentration-dependent variation in diffusion of the particle stream.

The nanoporous reservoir here has cavities in the order of magnitude of nanometres. The sample liquid may accordingly settle out in these cavities, such that the sample liquid is stored in the reservoir. Because the reservoir is nanoporous, it may accordingly have a large number of such cavities. As a result, in particular, a majority of the volume of the nanoporous reservoir can be used for uptake of the sample liquid. By virtue of the nanoporous cavities, the sample depot can preferably function as capillary pump and pump the sample liquid into the measurement chamber.

The measurement chamber can lead the sample liquid off into the sample depot.

This can achieve a constant flow of the sample liquid. In the case of such a constant flow of the sample liquid, i.e. without obstruction and without abrupt flow of the sample liquid, it is possible to achieve quantitative evaluation of the occurrence of the analyte(s) in a particularly simple manner.

The device may have a microelectronic circuit configured to receive the electrical signal from the cantilever pair.

The microelectronic circuit can, for example, measure the individual transducer states and/or pass the measurements onward to a control unit that converts the measurements to digital values and prepares them for a final device. The microelectronic circuit is accordingly an interface between the analogue measurement value production in the cantilever pair and the final device in which the measurement data evaluated.

For this purpose, the microelectronic circuit may especially comprise analogue/digital (A/D) converters.

The microelectronic circuit may be arranged outside the measurement chamber, especially around the measurement chamber.

This has the advantage that the cantilever pair can be arranged around the measurement chamber in any desired manner, and the electronic outputs from the transducers the microelectronic circuit can be kept as short as possible. This especially reduces electronic noise.

The microelectronic circuit may also be a deep embedded system capable of signal processing.

The microelectronic circuit may be connected to an antenna, in which case the antenna can run around the measurement chamber.

The antenna may consist here of one or more conductor tracks that can be conducted around the measurement chamber. The inductivity of the antenna can be adjusted by the size of the antenna and the number of conductor tracks involved.

For example, the microelectronic circuit may have a battery that can be inductively charged by the antenna.

The battery can provide, for example, the current and voltage supply of the transducers, such that the electrical resistance of the transducers can be measured. By virtue of the inductive charging, the apparatus additionally does not require a wired power connection, such that the device is particularly transportable. Instead of or in addition to a battery, it is possible to use an accumulator.

The microelectronic circuit may be read out via the antenna.

For example, the antenna may be used as a physical RFID or NFC (Near Field Communication) or WLAN or Bluetooth interface, such that the transducer measurements can be read out and evaluated at regular time intervals. For example, a device equipped with an NFC interface can be read out by smartphone if the reading antenna of the smartphone is brought close to the device antenna. The data may be intermediately stored if there is no communication connection, and exchanged when the communication connection has been re-established.

The cantilever pair may be formed from a chip.

This may mean that the sensor is produced on a semiconductor structure that permits further data processing of the transducer measurements or of the output value from the A/D converter logic unit. In particular, a chip may also mean what is called a system-on-a-chip, wherein all the functional units of the measurement system are formed integrally on a single electronic component.

The reference and test cantilevers may comprise Si₃N₄, SiO₂, Si₃N₄/SiO₂, SiC, Si or consist of Si or comprise a polymer or a metal with a thin oxide layer, for example aluminium oxide. The bases or the overall base may equally also comprise the materials mentioned. The bases and the reference and test cantilevers may also be produced in one piece collectively from the materials mentioned by conventional production processes as known in the processing of wafers.

The silicon-based reference and test cantilevers mean that it is possible to use production methods known from the semiconductor industry, thereby making it possible to produce sensors according to the invention on a large industrial scale. Polymers can likewise be produced on a large industrial scale and have the advantage that their material properties can be predetermined to a large degree.

The measurement chamber may take the form of a cavity in the chip.

This has the advantage that the chip provides both the cantilever pair and the measurement chamber volume. Accordingly, the mechanical connection between cantilever pear and measurement chamber is fixed, and the measurement chamber and the cantilever pair may be manufactured together, which can reduce production costs. In particular, it is possible here to achieve the effect in a particularly simple manner that the cantilever pair is arranged on the wall of the measurement chamber.

The chip may have application-specific integrated circuits.

Application-specific integrated circuits (“ASICs”) may assume a predefined function here of converting the input value from the transducers with the given function to an output value.

It is preferably thus also possible to integrate further sensors, for example gyroscopes, temperature sensors and optical sensors for determination of blood gas values and other parameters such as pulse rate.

The chip may have a multitude of cantilever pairs that are especially arranged around the cavity, where each cantilever pair is specific for the detection of an analyte.

For example, a first cantilever pair can measure the lactate value, a second cantilever pair can measure the blood oxygen value, a third cantilever pair can measure an inflammation value, a fourth cantilever pair can measure a further biomarker, and so forth. This achieves the effect that a multitude of analytes is determined simultaneously in the sample liquid, such that a single device can effect a complex analysis of the vital state of the sample body.

The chip may be arranged on the microelectronic circuit. In particular, the transducers may be connected to the application-specific integrated circuits via bond wires. In particular, the components of the microscopic circuit may be manufactured in a first manufacturing step, and the chip can be produced separately therefrom. Thus, the electronic evaluation and the measurement recording may be optimized independently.

The sample inlet area of the measurement chamber may have a multitude of capillaries.

This can achieve improved and preferably controlled inflow of the sample liquid into the measurement volume. In addition, by means of a multitude of capillaries, it is possible to better mount the device on the sample body and, for example, to prevent lateral slippage, such that irritation to the skin is avoided.

The abovementioned object is also achieved by a sensor device for the detection of an analyte in a sample having the features of claim 21. Accordingly, a sensor device for the detection of an analyte in a sample is proposed, where the above-described device is arranged in an emplastrum, where the emplastrum is bonded to a layer, and the capillary penetrates into the layer and/or penetrates through the layer, where it takes a sample and feeds it to the measurement chamber.

An emplastrum here is a pressure-sensitively adhesive layer by which the device can be secured on the layer of the sample body. For example, the layer may be skin, preferably the skin of a land animal, more preferably the skin of a mammal, most preferably the skin of a human. It may also be the case that the sample body is the body of a fish.

The device may be arranged in a pocket of the emplastrum, such that the sample inlet area runs parallel to the pressure-sensitively adhesive layer of the emplastrum. The sample inlet area is exposed here, such that the capillaries are not concealed by the pocket of the emplastrum.

Such a configuration allows the device to be held reliably on the sample body, while the sample liquid is withdrawn and analysed.

For example, the measurement electronics with battery and/or accumulator may be releasably connected to the measurement chamber and the capillary. This allows the measurement chamber together with the capillaries to be exchanged for reasons of hygiene, while the measurement electronics can be used further and/or can continue to be used.

The diagonal of the sensor device may be between 10 mm and 15 mm, and the height may be between 2 mm and 3 mm.

By virtue of the small form factor, it is possible that the user, for example a human, can wear the sensor device in an inconspicuous manner and experiences a high degree of wear comfort. The movements of the human or animal are restricted here only to a minor degree, if at all.

It is thus preferably possible to prevent the sensor device from sticking to clothing, and only a comparatively small skin area is irritated, which means, for example, that it is possible to minimize any occurrence of pruritus.

The device and especially the at least one capillary may be sealed by a removable, in particular water-soluble, sealing layer which is dissolved for the first time prior to the application of the emplastrum. In this way, in particular, the capillary is kept clean, clear and permeable during transport.

### BRIEF DESCRIPTION OF THE FIGURES

Exemplary embodiments of the invention are elucidated in detail by way of the description of the figures that follows. The figures show:

FIG. 1 is a schematic diagram of the device according to an exemplary aspect of the invention;

FIGS. 2A, 2B, 2C, 2D, and 2E are schematic diagrams of a cantilever pair and its mode of function;

FIG. 3 is a schematic diagram of the device according to an exemplary aspect of the invention;

FIGS. 4A and 4B are schematic diagrams of the sensor device according to an exemplary aspect of the invention;

FIG. 5A, B are further diagrams of the sensor device according to an exemplary aspect of the invention; and

FIG. 6 a schematic diagram of the use of a device according to an exemplary aspect of the invention in/on a food package.

### DETAILED DESCRIPTION

Preferred embodiments are described below with reference to the figures. Elements that are identical, similar or have the same effect are given the same reference numerals here in the various figures, and repeated description of these elements is omitted in some cases in order to prevent redundancy.

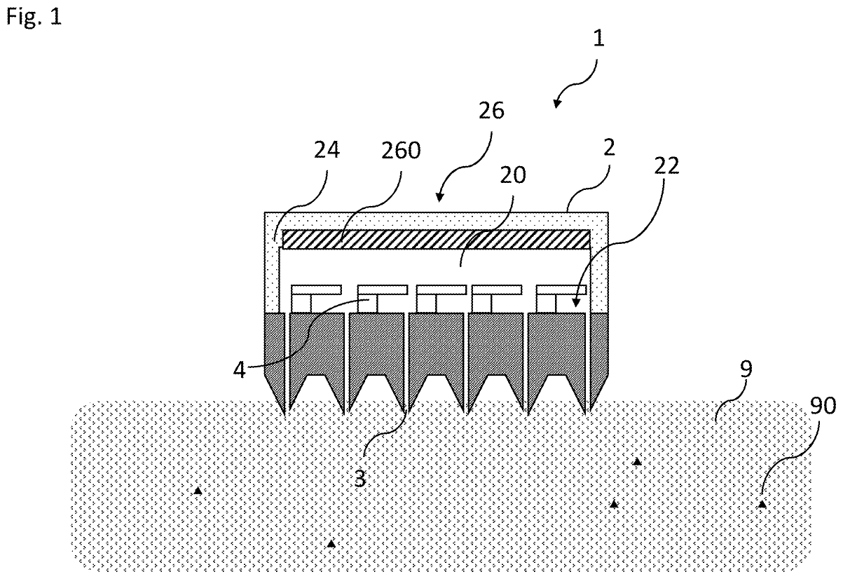

FIG. 1 shows a schematic of an device 1 according to the invention. The device 1 comprises a measurement chamber 2 that has a measurement volume 20 bounded by a sample inlet area 22, a measurement chamber wall 24 and a measurement chamber lid 26.

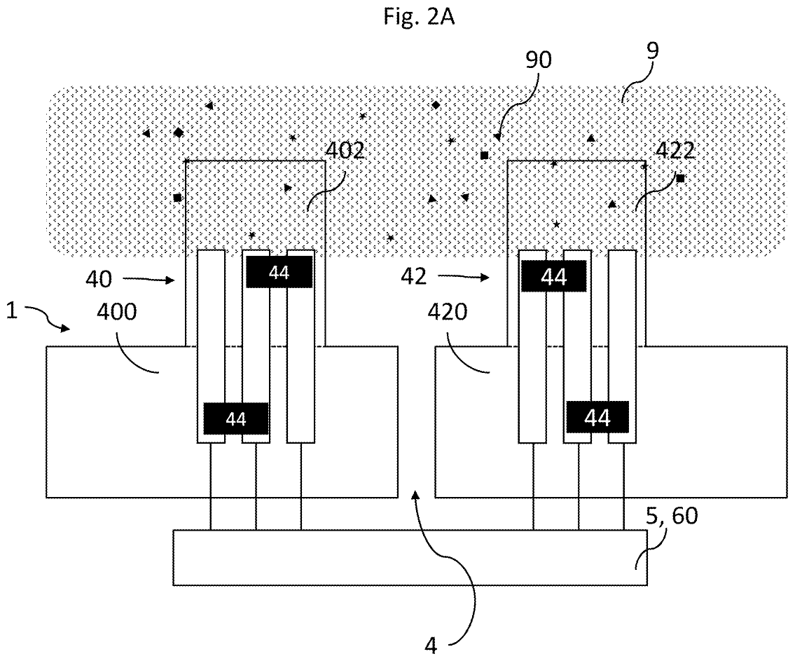

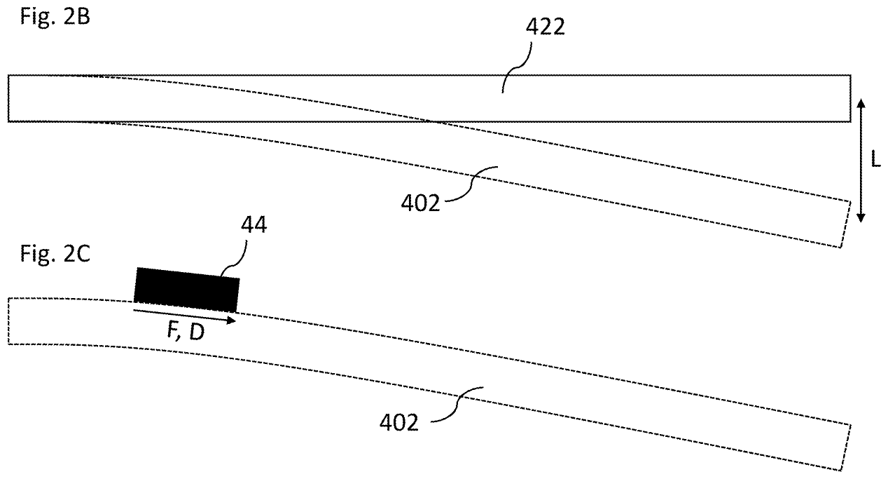

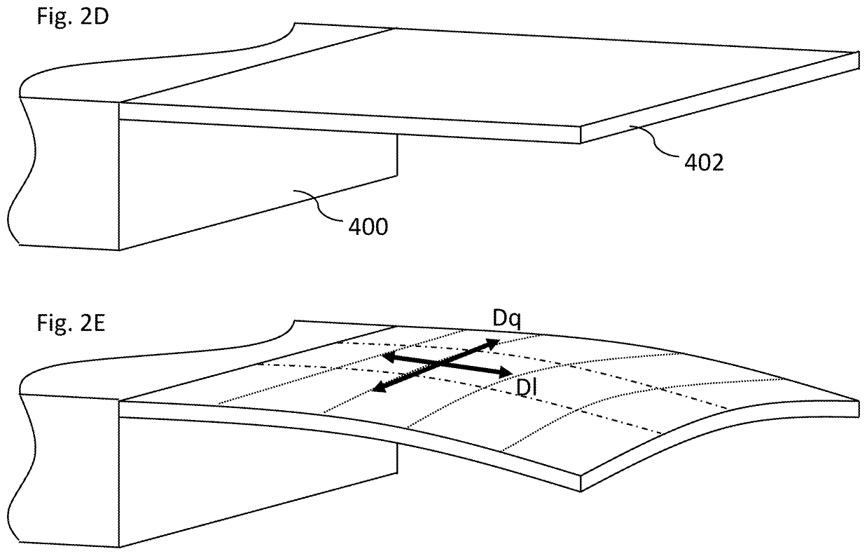

Introduced within the sample inlet area 22, are multiple capillaries 3, in the form of microneedles, that can take a sample liquid 9 from a sample body and transport it into the measurement chamber volume 20. Arranged within the measurement chamber volume 20 are various cantilever pairs 4 that comprise both a reference cantilever 42 and a test cantilever 40 (shown for example in FIGS. 2A to 2E) and are described in detail further down.

The capillaries 3 guide the sample liquid 9 to the cantilever pairs 4, where the analyte 90 in the sample which is present in the sample liquid 9, indicated here in very schematic form, causes specific bending or variation in the surface tension of the cantilevers 40, 42 (see, for example, FIGS. 2A to 2E), depending on whether or not the analyte 90 can interact with the test cantilever 40. This bending or variation in surface tension, by means of strain gauges or by means of transducers 44, can generate an electrical measurement signal such that the relative bending of the cantilevers 40, 42 can be detected and hence an occurrence of an analyte 90 can be detected.

In addition, FIG. 1 shows an optional sample reservoir 260, which, in this embodiment, is arranged on the measurement chamber lid 26. The sample reservoir 260 is capable here of taking up the sample liquid 9 from the measurement chamber 2 and storing it. In particular, the sample depot can serve as pump for the sample liquid via the nanopores. In this way, the sample liquid 9 can be exchanged within the measurement chamber volume 20, such that a constant liquid flow of new sample liquid 9 can be fed to the at least one cantilever pair 4. Accordingly, time-dependent and quantitative and qualitative detection of the analyte 90 is possible.

The capillary 3, which may be used for continuous inflow of sample liquid 9 from the sample body into the measurement chamber volume 20, guides a constant replenishment of sample liquid 9 into the measurement chamber volume 20 via diffusion forces and/or capillary forces. In order to adjust the strength and size of the inflow, the number of capillaries here 3 in the sample inlet area 22 may be adjusted. Typically, it is also possible to transport more sample liquid 9 into the measurement chamber volume 20 via multiple capillaries 3.

FIGS. 2A to 2E show the fundamental mode of function of the cantilever pair 4. The cantilever pair 4 in FIG. 2A comprises a test cantilever 40 that has a base 400 and a deformable part 402, and a reference cantilever 42 that has a base 420 and a deformable part 422. Arranged on the cantilevers 40, 42 are transducers 44 that convert the bending of the cantilevers 40, 42 to an electrical measurement signal by interaction with the analyte 90. This electronic measurement signal can be registered and processed further, for example, by means of the microelectronic circuit 5 and/or the application-specific integrated circuits 60.

The device 1 has the function of indicating the occurrence and preferably the amount of an analyte 90 in a sample 9. In any case, the device 1 is to be used to examine the sample 9 for the occurrence and/or a concentration and/or an amount of the analyte 90. For this purpose, a receptor layer is applied to the test cantilever 40, with which an analyte 90 can interact, or a receptor layer that can adsorb or absorb the analyte 90.

If the sample 9 includes an analyte 90, this analyte may thus interact with the receptor layer. This may lead to the surface tension of the section, coated with the receptor layer, of the deformable part 402 of the test cantilever 40 changing, which leads to a deformation of the deformable part 402 of the test cantilever 40.

However, even the interaction with the sample liquid 9 with the cantilevers 40, 42 can result in registration of a deformation, for example in that merely the surface tension of the sample liquid 9 acts on and deforms the deformable part 402 of the test cantilever 40. The presence of an analyte 90 is accordingly not responsible for such a deformation.

In order to establish the magnitude of this basic effect of the sample 9 on the test cantilever 40, the reference cantilever 42 is brought into contact with the sample 9 at the same time as the test cantilever 40. For this purpose, the reference cantilever 42 has a reference layer, with which an analyte 90 cannot interact, or a reference layer that is not able to adsorb or absorb the analyte 90. In this case, any interaction solely with the analyte 90 should be avoided in order to enable differentiation with regard to the measured signal from the test cantilever 40.

Since both the test cantilever 40 and the reference cantilever 42 interact with the sample 9, both cantilevers 40, 42 interact similarly with the sample 9. The difference in this case is however that the test cantilever 40 is additionally able to interact with any analyte 90 present via its reference layer. There will accordingly be a difference in the measured signals from the transducers 44 if an analyte 90 occurs in the sample 9. The magnitude of the difference between the measured signals can accordingly, in the simplest case, be used to infer the amount of the occurrence of the analyte 90 in the sample 9.

In order to work out the difference merely by measurement technology, the transducers 44 on the deformable parts 402, 422 and the bases 400, 420 of the cantilevers 40, 42 may be connected in a full bridge, such that the electronic measurement signal consists in a comparison of the ratios of the resistance values of the transducers 44.

FIG. 2B shows the comparison of the deformable parts 402, 422 of the reference and test cantilevers 42, 40 (see FIG. 2A) in the event of a deformation and longitudinal extension. The deformable part 422 of the reference cantilever 42 has an upper surface and a lower surface. The deformable part 402 of the test cantilever 40 likewise has an upper surface and a lower surface. If an analyte 90 of the sample 9 interacts with the test cantilever 40, or with the receptor layer, the deformable part 402 deforms from the fixed part (that transitions into the base of the test cantilever) toward the freely mobile part of the deformable part 402. The deflection L shown here results from the relative deflection between the deformable part 422 of the reference cantilever 42 and the deformable part 402 of the test cantilever 40 due to the interaction with the analyte 90.

The deformation of the deformable part 402 of the test cantilever 40 is shown in FIG. 2C. The cause of this is that the upper surface and the lower surface of the test cantilever 40 extend to different degrees. Because of the great extent D at the upper surface, a transducer applied thereto can register an extension force F. The registered extension force F can be converted here to an electronic signal by the test transducer 44 or influence an existing electronic signal, for example an applied voltage. This can be accomplished, for example, in that the resistance of the transducer 44 changes if it experiences an extension force F, which in turn results in an extension of the transducer 44.

The transducer 44 would also be able to detect a contraction of the surface on which it is arranged. In the embodiments shown, the transducers, however, are always arranged on surfaces where extension is expected, i.e. on the upper surfaces and/or the lower surfaces in particular.

The different surface tensions on the lower side and the upper side of the cantilever accordingly result in the described deformation or extension of the cantilever.

In particular, the alignment of the transducers relative to the alignment of the cantilevers 40, 42 plays an important role. FIG. 2D shows a non-deflected cantilever 40 for example. If the cantilever 40 comes into contact with the analyte 90, the surface tension changes and there is deformation of the deformable part 402 of the cantilever 40. The deformable part 402 thus undergoes, for example, deformation perpendicular to the base 400 as shown in FIG. 2E. This is accompanied by a longitudinal extension DI of the upper surface. At the same time, deformation takes place parallel to the base 400, or to the bending edge, which is accompanied by a transverse extension Dq of the upper surface. The geometry of the cantilever 40 makes it possible to determine the direction in which a greater extension D is brought about. The transducer may in particular be aligned in this direction in order to generate a particularly large measurement signal.

By virtue of an increased mechanical extension at the site of the transducer 44, it is possible to even further improve the signal found by the transducer 44. Such oversizing can be achieved, for example, via the arrangement and shape of the electrodes.

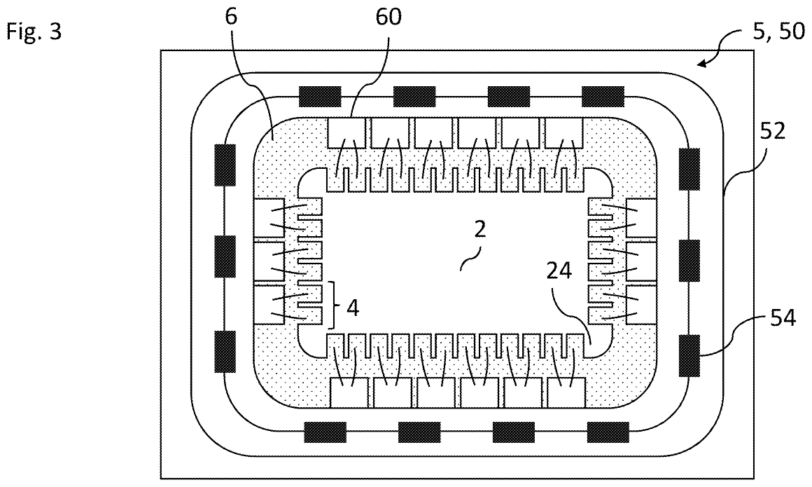

FIG. 3 shows a top view of an embodiment of the device 1. The cantilever pairs 4 are formed here on a common chip 6. In addition, application-specific integrated circuits 60 have been applied atop the chip. In particular, a multitude of cantilever pairs 4 are formed within the chip 6 and arranged around a cavity. The cavity is bounded at the edge by the measurement chamber wall 24, which is formed, for example, by the chip 6. Thus, the chip 6 provides not only the cantilever pairs 4 but also the measurement chamber walls 24 by which the measurement chamber volume is bounded. In addition, the cantilever pairs 4 are arranged on or at the measurement chamber wall 24.

The chip 6 is arranged on the microelectronic circuit 5 which is manufactured, for example, from a printed circuit board 50 (PCB). Printed circuit boards can be produced, for example, in a simple manner by a photolithography and in large numbers.

The microelectronic circuit 5 contains further microelectronic elements 54, for example a battery or a processor and a memory for storage of measurements. The microelectronic circuit 5 likewise has conductor tracks that connect the microelectronic elements 54 to one another. The microelectronic circuit 5 is arranged around the chip 6 and especially around the measurement chamber volume 20 (see FIG. 1). In addition, the microelectronic circuit 5 has an antenna 52, via which, for example, the battery of the microelectronic circuit 5 can be charged, or else can be utilized for communication with the processor and the memory. For example, the antenna 52 can read out the analysis measurements from the cantilever pairs 4.

It is also possible that a reading device induces a current in the antenna 52 that provides the measurement current of the transducers 44. In that case, the device 1 can be used without batteries. Such a principle is known, for instance, in the area of Near Field Communication (NFC), in which, for example, the data from (battery-free) credit cards is issued via a circuit which is supplied with energy via a voltage induced by the reading device. This has the advantage that the device 1 does not require its own energy source and is only “woken up” when a reading device is indeed provided to read out the analysis.

Alternatively or additionally to the configuration shown in FIG. 3, in which all cantilever pairs 40, 42 are arranged of a common chip 6, individual cantilever pairs 40, 42 may also be arranged on separate chips 6. Correspondingly, as many chips as cantilever pairs 40, 42 may be provided, which are then arranged around the central cavity.

The chip 6 or the chips may also be kept within a receptacle in the measurement chamber wall 24, in which case the chip(s) do not form the measurement chamber wall, and this is instead formed by a separate housing.

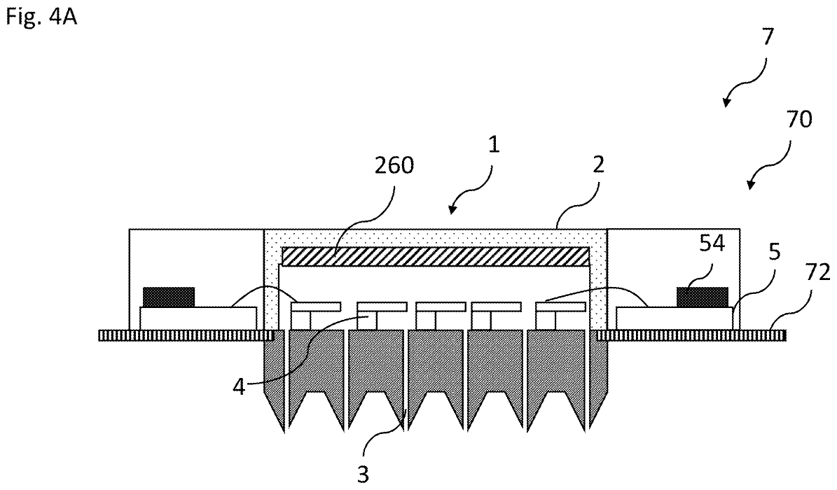

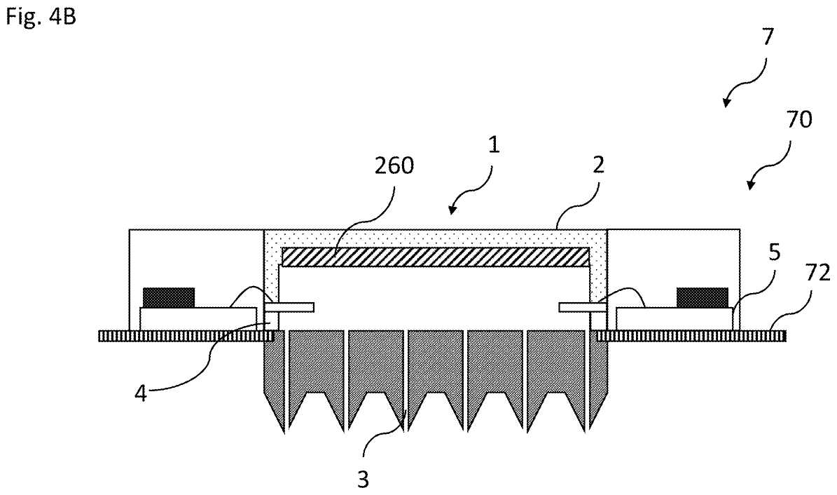

FIG. 4A shows a sensor device 7 comprising a device 1. An emplastrum 70 of the sensor device 7 has a pressure-sensitively adhesive layer 72. This pressure-sensitively adhesive layer 72 may be bonded to a sample body, for example to the skin of a human. The capillaries 3 penetrate into the skin, for example up to the dermis of the skin, and can guide a sample liquid 9 (see FIG. 1) in the form of a body fluid, for example lymph or interstitial fluid or perspiration, therefrom into the measurement chamber 2. Arranged within the measurement chamber 2 are a multitude of cantilever pairs 4 which analyse the body fluid 9 for particular analytes 90. The emplastrum 70 here encloses the entire device 1 including the microelectronic circuits 5 and the microelectronic components 54. The cantilever pairs 4 are bonded here to the microelectronic circuit 5 via bond wires, for example.

FIG. 4B shows the sensor device 7, except that the cantilevers are arranged at the measurement chamber wall 24 (FIG. 3).

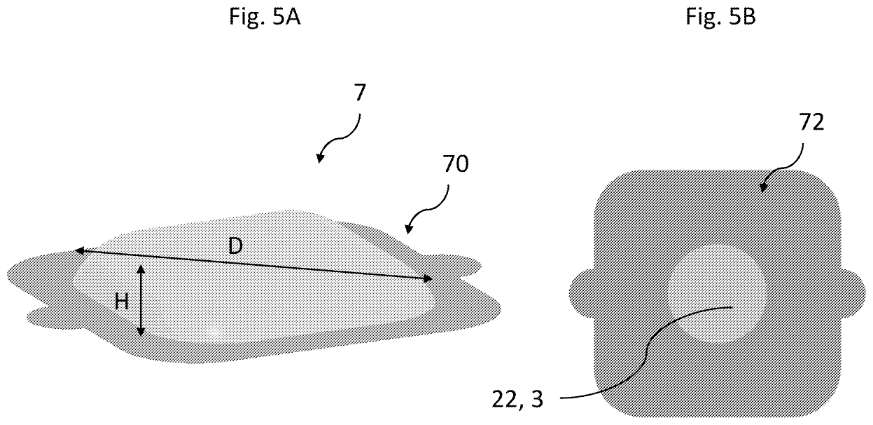

FIG. 5A shows a schematic outer view of the sensor device 7. In particular, the emplastrum 70 that encompasses the device 1. In particular, the emplastrum 70 can seal the device in a watertight manner, such that the device is not damaged by liquids in coming from the outside, for example shower or bath water or rain.

The diagonal D of the sensor device here may, for example, be between 10 mm and 15 mm and the height H may, for example, be between 2 mm and 3 mm, in order to assure comfortable wearing of the sensor device. In addition, the edges may be rounded, such that the emplastrum has a flat construction and the emplastrum does not stick to clothing.

FIG. 5B correspondingly shows the underside of the sensor device 7. The capillaries 3 of the sample inlet area are accessible from the outside, such that bonding of the emplastrum 70 on the skin by the bonding area 72 results in penetration of the capillaries 3 into the skin, in order to take sample liquid 9 therefrom and feed it to the measurement chamber volume 20.

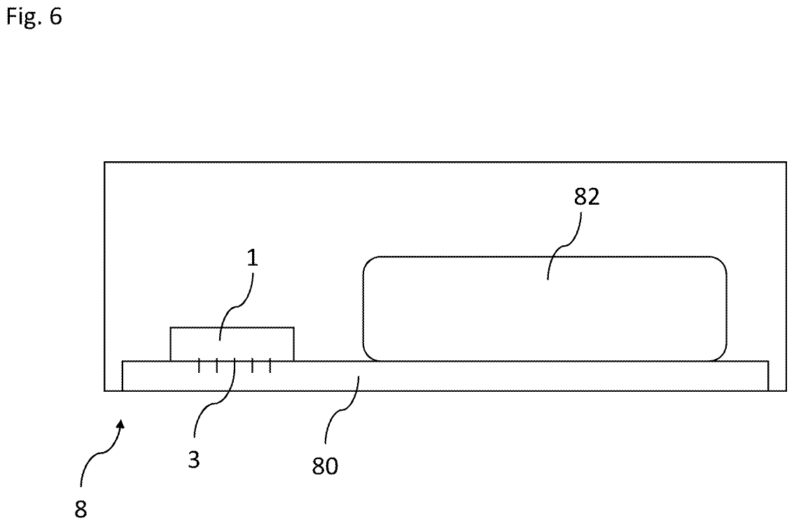

FIG. 6 shows a further field of application of the device 1. For example, the device 1 can be used in the foods industry, for example in food packages 8. The package 8 encloses the food 80 arranged, for example, on an absorbent inlay 82, in order to collect liquids that escape, for example meat juice, vegetable juice or fruit juice. Such foods often have a short shelf life, such that rapid consumption is indispensable, in order to avoid any possible endangerment to health. The device 1 makes it possible to collect the juice that escapes by means of the absorptive inlay 80 and to feed it via the capillary 3 to the device 1 and to analyse it and to examine it, for example, for analytes such as fungi or microbes.

Where applicable, all individual features that are illustrated in the working examples may be combined with one another and/or exchanged for one another without departing from the scope of the invention.

### LIST OF REFERENCE SIGNS

1 device
2 measurement chamber
20 measurement volume
22 sample inlet area
24 measurement chamber wall
26 measurement chamber lid
260 sample depot
3 capillary
4 cantilever pair
40 test cantilever
400 base
402 bendable part
42 reference cantilever
420 base
422 bendable part
44 transducer
5 microelectronic circuit
50 PCB
52 antenna
54 microelectronic components
6 chip
60 application-specific integrated circuit
7 sensor device
70 emplastrum
72 bonding area
8 package
80 absorbent inlay
82 food
9 sample
90 analyte

1. A device for detection of at least one analyte in a sample, the device comprising:
at least one measurement chamber;
at least one capillary configured to obtain a sample and to feed the sample to the at least one measurement chamber; and
at least one cantilever pair including a reference cantilever and a test cantilever,
wherein the measurement chamber is configured to collect the sample and provide the sample it to the at least one cantilever pair,
wherein the test cantilever is configured for selective uptake of the analyte from the sample and the reference cantilever is configured for selective non-uptake of the analyte from the sample,
wherein the selective uptake of the analyte by the test cantilever and the selective non-uptake by the reference cantilever causes at least one of a relative deformation and a relative change in a surface tension of the test cantilever relative to the reference cantilever, and
wherein the at least one of the relative deformation and the relative change in the surface tension of the test cantilever and the reference cantilever results in detection of the analyte.

2. The device according to claim 1, wherein the detection of the analyte in the sample is conducted continuously in a time-resolved manner.

3. The device according to claim 1, wherein the analyte is a biomarker and the sample is a body fluid.

4. The device according to claim 1, wherein the at least one capillary is configured to feed the sample to the at least one measurement chamber via at least one of diffusion forces and capillary forces.

5. The device according to claim 1, wherein the at least one capillary has a diameter of less than 200 μm and a length between 500 μm and 5000 μm.

6. The device according to claim 1, wherein the at least one capillary is a microneedle.

7. The device according to claim 1, wherein the at least one measurement chamber encloses a measurement volume that is defined by a sample feeding area having the at least one capillary, adjoined by at least one measurement chamber wall, and a measurement chamber lid.

8. The device according claim 7, wherein the at least one cantilever pair is arranged on or in the measurement chamber wall.

9. The device according to claim 1, wherein the at least one measurement chamber has a nanoporous reservoir as a sample inlet area that is configured to take up the sample and function as a pump for the sample.

10. The device according to claim 1, wherein the sample inlet area comprises a multitude of capillaries.

11. The device according to claim 1, further comprising a microelectronic circuit configured to receive an electrical signal from the at least one cantilever pair.

12. The device according to claim 11, wherein the microelectronic circuit is arranged outside the measurement chamber and around the measurement chamber.

13. The device according to claim 11, wherein the microelectronic circuit is connected to an antenna that runs around the measurement chamber.

14. The device according to claim 13, wherein the microelectronic circuit has at least one of a battery and an accumulator that is configured to be charged inductively via the antenna.

15. The device according to claim 13, wherein the microelectronic circuit is configured to be read out via the antenna.

16. The according to claim 1, wherein the at least one cantilever pair is formed from or on a chip.

17. The device according to claim 16, wherein the measurement chamber comprises a cavity in the chip.

18. The device according to claim 16, wherein the chip has application-specific integrated circuits.

19. The device according to claim 17, wherein the chip has a multitude of cantilever pairs that are arranged around the cavity, where each cantilever pair is configured for the detection of an analyte.

20. The device according to claim 16, wherein the chip is arranged on the microelectronic circuit.

21. A sensor device for detection of an analyte in a sample, the sensor device comprising:
the device according to claim 1,
wherein the device is arranged in an emplastrum that is configured to be bonded to a layer such that the at least one capillary penetrates into at least one the layer and through the layer, and
wherein the at least one capillary is configured to obtain the sample and feeds the sample to the measurement chamber.

22. The sensor device according to claim 21, wherein the layer is one of skin of a living creature or a food package.

23. The sensor device according to claim 21, wherein a diagonal of the sensor device is between 10 mm and 15 mm and a height is between 2 mm and 3 mm.
